(19) United States
(12) Patent Application Publication
(10) Pub. No.: US 2025/0143354 A1
(43) Pub. Date: May 8, 2025

(54) A TWO-STEP PROCESS AND SYSTEM FOR PREPARING A LIQUID OAT BASE OR DRINK FROM AN OAT DERIVED MATERIAL

(71) Applicant: Oatly AB, MALMÖ (SE)

(72) Inventors: David HELLBORG, MALMÖ (SE); Joakim BJUNÖ, MALMÖ (SE); Karin PETERSSON, MALMÖ (SE); Sofia EHLDE, MALMÖ (SE)

(21) Appl. No.: 18/726,096

(22) PCT Filed: Dec. 30, 2021
(86) PCT No.: PCT/EP2021/087835

§ 371 Date: Jul. 1, 2024

(51) Int. Cl. A23L 2/38; A23L 7/104; C12M 1/00; C12M 1/02; C12N 9/26; C12N 9/80

(52) U.S. Cl. CPC A23L 2/38; A23L 7/107; C12M 23/58; C12M 41/18; C12N 9/2414; C12N 9/2425; C12N 9/80; C12Y 302/01001; C12Y 302/01002; C12Y 305/01044

(57) ABSTRACT

The invention relates to a process for preparing a liquid oat base or drink from an oat derived material. The process comprises a first enzymation of the oat derived material, heating the oat derived material to terminate the enzymation thereof, and a second enzymation of the oat derived material. The first and/or the second enzymation comprises contacting the oat derived material with starch degrading enzyme, and the first and/or second enzymation further comprises contacting the oat derived material with protein solubilizing enzyme.

The invention also relates to a system for preparing a liquid oat base or drink from an oat derived material.

### TECHNICAL FIELD OF INVENTION

The invention relates to a process and system for preparing a liquid oat base or drink from an oat derived material.

### BACKGROUND

There is a growing interest in refraining from consumption of dairy products and from meat products, for example for health reasons, religious reasons, and sustainability reasons. In addition, there is a growing trend to choose products for consumption in the daily life which contribute to a positive climate impact. This may mean excluding or at least reducing intake of dairy based products as well as meat products.

Oat is well known for containing nutritious compounds and may be considered a sustainable vegetable ingredient in food production. Oat in different forms is therefore an often-used ingredient in healthy and nutritious foods.

For example, use of oats in production of oat-containing aqueous liquid, sometimes called an oat base, is well known. Such oat base may be used as ingredient or base for manufacturing of different food products, for example, analogues for dairy products. Oat beverage, such as an oat drink is one well known example.

Manufacturing of oat base, and oat drink, typically involves subjecting micronized oats to one starch degrading enzymatic treatment in aqueous suspension. Process parameters, such as temperature, have an effect on both enzymes and the micronized oats of the suspension. It is challenging and problematic during the manufacturing to maintain the micronized oats in the aqueous suspension, in a form suitable for the enzymatic treatment. It is further problematic to control the enzymatic activities, as well as maintaining nutritious qualities from the oats, during the manufacturing process.

There is, thus, a need for providing improved control of manufacturing process for the oat base, while at the same maintaining control of the quality of the oat base.

### SUMMARY OF INVENTION

It is an objective of the present invention to, at least partly, improve the overall process of preparing an oat base or drink.

According to a first aspect of the invention, these and other objects are achieved, in full or at least in part, by a process for preparing a liquid oat base or drink from an oat derived material. The process comprises a first enzymation of the oat derived material, heating the oat derived material from the first enzymation to terminate the enzymation thereof, and a second enzymation of the oat derived material from the heating, wherein the first and/or the second enzymation comprises contacting the oat derived material with starch degrading enzyme, and wherein the first and/or second enzymation comprises contacting the oat derived material with protein solubilizing enzyme.

Thereby, the liquid oat base or drink having an improved soluble oat protein content may be provided. A desirable and synergistic effect, which improves levels of solubilized proteins, is achievable from the combination of using starch degrading enzymes and protein solubilizing enzymes.

Enzymation of the product in two separate steps will make it possible to provide an oat base or drink with for example a more even consistent quality.

After the first step of enzymation the oat derived material is allowed to be subjected to higher temperatures of the heating compared to a process without a first step of enzymation. The use of higher heating temperature is believed to be, at least in part, made possible due to a result of starch degradation in the first step of enzymation. The starch degradation results in lower degree of gelatinization during the heat treatment as compared to heat treatment without a preceding first step of enzymation.

The possibility of increasing the temperature in the heating step following the first step of enzymation will further improve the effect that the starch of the oat derived material swells and, as a result thereof, the starch will be available to the present enzymes at a higher extent in the second enzymation step.

The higher probability of availability of starch to be degraded by the second enzymation will lead to a higher output of oat base or drink, for example due to a higher degradation of starch as compared to only using one enzymation step. The combination and order of the first step of enzymation with the following heat treatment and second enzymation is therefore considered advantageous.

If the heat treatment would be performed prior to or parallel with the first enzymation, the swelling of the starch would occur at a higher degree and may likely result in considerably reduced or even absence of enzymation unless specific high heat tolerant enzymes were used.

With the first enzymation being performed prior to the heating, the enzymation may be performed on an oat derived material with suitable properties for the enzymation and/or the process, such as, for example, lower viscosity as well as provide an oat derived material which will not swell as severely in the following heat treatment. The heating following this first enzymation thereby allows for consecutive enzymation of partly enzymatically treated and partially swollen starch from the oat derived material, as discussed above. The heating further allows for deactivation of enzymes of the first enzymation, which may be beneficial for further treatment of the oat derived material without any or low active effect of the enzymes of the first enzymation such as for example the use of other enzyme(s) in the second enzymation with other desired enzymation properties.

The first enzymation of the oat derived material may be a first enzymation of oat derived material in aqueous suspension, wherein a first enzymatically treated suspension is formed.

The heating the oat derived material from the first enzymation to terminate the enzymation thereof, may be heating the first enzymatically treated suspension, wherein a heat-treated suspension is formed.

The second enzymation of the oat derived material from the heating may be a second enzymation of the heat-treated suspension, wherein a second enzymatically treated suspension is formed. The second enzymatically treated suspension, may, with or without further treatment, be an oat base or drink.

Thus, the second enzymation may, optionally, be followed by further treatment. The further treatment may be, for example heating and/or removal of particulate matter or compounds such as for example fiber residues or other non-desired matter originating from the oat derived material after the second enzymation. For example, the further treatment may be sedimentation, decantation, and/or filtering.

The process may further comprise cooling the oat derived material after the step of heating the oat derived material and before the step of the second enzymation of the oat derived material.

The process may further comprise a step of steaming the oat derived material after the step of heating the oat derived material to terminate the enzymation and before the step of cooling the oat derived material.

The process may further comprise a step of heating the oat derived material a second time to terminate the enzymation thereof, after the step of the second enzymation of the oat derived material.

The process may further comprise a step of cooling the oat material a second time after the step of heating the oat derived material the second time.

The process may further comprise a step of steaming the oat derived material a second time, after the step of heating the oat derived material the second time and before the step of cooling the oat derived material the second time.

The oat derived material may comprise or be in form of micronized oats or micronized oat-kernels, or oat-flour, or fractions of oat. For example, the oat derived material may, thus, be in form of milled, crushed or ground oat derived material, or a combination thereof. As another example, the fractions of oat may be oat bran or fiber reduced oat flour.

The oat derived material may be a mixture of oat and water. For example, the oat derived material may be a mixture of oat and water an aqueous or water-based suspension or slurry of one or more of micronized oat-kernels, oat-flour, and/or fractions of oat.

If the oat derived material comprises or is in form of micronized oats or micronized oat-kernels, or oat-flour, or fractions of oat, the oat derived material may be oat derived material provided for or entering the first enzymation. Such oats, for example micronized oats, will in the process be at least partially degraded or transformed, for example by means of the first and second enzymation, and it shall be understood that as it progresses in the process it may cease to be in the provided form of micronized oats or oat-kernels.

The oat derived material may be selected from heat-treated wet milled oats, heat-treated dry milled oats, heat-treated oat bran, heat-treated dehulled or hulled/naked dry milled oat flour.

The oat derived material may be provided as an aqueous or water suspension, slurry or paste. Thereby, the oat derived material can be pumped and mixed and efficiently subjected to heating. Further, such oat derived material may be efficiently enzymatically treated. The content of oat derived material in water may be, for example, 5-30% by weight dry substance, such as 10-25% by weight dry substance based on the weight of the oat derived material in water.

The oat derived material comprising oat starch may be subjected to a partial hydrolysis by a starch degrading enzyme, such as amylase. The amylase may be one or more of α-amylase and β-amylase.

The oat derived material comprising proteins may be subjected to a protein solubilizing enzyme such as a protein deamidase, preferably protein glutaminase.

Other enzymes for solubilizing proteins may be used than the protein deamidase or protein glutaminase. Solubilization of proteins, as used herein, is intended to describe an increase of solubility of the proteins in water or aqueous liquid as compared to prior to or without the treatment, such that at least a portion of the proteins present in the oat derived material may be provided in aqueous or water solution. Solubilization may be realized, for example, by enzymes modifying amino acids of the proteins, such as side chains of amino acids, into more hydrophilic form, for example by modifying amide groups of the side chains. Any type of enzymes suitable for this purpose in providing an oat base or oat drink may be considered for the purpose. Converting the proteins into a more water-soluble form, or solubilizing proteins, allows for a more efficient process for preparing a liquid oat base or drink from an oat derived material, as well as improved properties of the oat base or drink. Increase solubility may, for example, improve extraction of proteins from an oat derived material matrix during the process, thus increasing levels of oat protein in the oat base or drink.

According to a second aspect of the invention, these, and other objects, and/or advantages that will be apparent from the following description, are achieved, in full or at least in part, by a system for preparing a liquid oat base or drink from the oat derived material. The system comprises a first enzymation section adapted for a first enzymation of the oat derived material, a heating device fluidly connected to and arranged downstream of the first enzymation section and adapted to heat the oat derived material, and a second enzymation section fluidly connected to and arranged downstream of the heating device and adapted for a second enzymation of the oat derived material from the heating device.

The heating device may be adapted to both heat the oat derived material and thereafter cool the oat derived material.

The heating device may comprise a heat exchanger and/or a steam injector.

The system may further comprise a second heating device fluidly connected to and arranged downstream of the second enzymation section and adapted to heat the oat derived material a second time.

The second heating device may be adapted to both heat the oat derived material in a first step and thereafter cool the oat derived material in a second step.

The second heating device may comprise a heat exchanger and/or a steam injector.

The first enzymation section and the second enzymation section may comprise a conduit or a reservoir, such as a tank, respectively.

Effects and features of the second aspect of the present invention are largely analogous to those described above in connection with the first aspect of the inventive concept. Examples mentioned in relation to the first aspect of the present invention are largely compatible with the further aspects of the invention.

Other objectives, features and advantages of the present invention will appear from the following detailed disclosure, from the attached claims, as well as from the drawings. It is noted that the invention relates to all possible combinations of features.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of the element, device, component, means, step, etc., unless explicitly stated otherwise.

The oat base or drink material may comprise, by dry weight, 5-20% protein, and 3-15% fat, and optionally 10-80% carbohydrates, including sugars. Typically, the oat base or oat drink may comprise sugars 2-8%, fibers 0.2-3% (out of which 0.1-1% of the oat drink are beta glucans), fat 0.0-2.5%, and proteins 0.5-2.5%, by weight of the oat base or drink. The liquid oat base or drink may comprise an oat derived material with 5-20% by weight dry substance, based on the weight of the liquid oat base or drink.

Herein is disclosed a process for preparing the liquid oat base or drink from the oat derived material. It shall be understood that the oat derived material changes in the process. In the present disclosure, in an attempt to improve understanding and clarity of the disclosure, the term oat derived material is sometimes used to describe and refer to oat derived material having undergone different treatment in the process, and, thus, one oat derived material may not be the same as another oat derived material. Sometimes herein, if deemed to improve clarity, the term oat derived material is further specified, for example, by defining it as an oat derived material from the first enzymation, or an oat derived material from the heating etc. Some examples of changes, which the oat derived material may undergo in the process, will now be given. Oat derived material in the first enzymation and the second enzymation may, for example, be subjected to starch degrading enzymes, which enzymes may degrade starch present in the oat derived material and, thus, change the oat derived material. As a further example, the heating the oat derived material may involve swelling of starch in the oat derived material, thus changing the oat derived material. To further exemplify, oat derived material early in the first enzymation may be micronized oat kernels with essentially intact starch, while oat derived material at the end of the first enzymation may be oat derived material comprising degraded starch.

The oat base or drink from the process may be mixed with additional additives and may then be referred to a liquid oat composition.

Herein is disclosed a process for preparing the liquid oat base or drink from the oat derived material by contacting the oat derived material with the enzyme such as the starch degrading enzyme, and the protein solubilizing enzyme. It shall be understood that the contact may be performed in many different ways such as for example by mixing the oat derived material and the enzyme in a supply or vessel such as a tank and/or a pipe. The mixing may be performed by any known mixing techniques such as in a mixing tank comprising impellers or blades.

As used herein, the term “comprising”, and variations of that term are not intended to exclude other additives, components, integers, or steps.

### BRIEF DESCRIPTION OF THE DRAWINGS

The above, as well as additional objects, features and advantages of the present invention, will be better understood through the following illustrative and non-limiting detailed description of examples of the present invention, with reference to the appended drawings, where the same reference numerals may be used for similar elements, and wherein:

FIG. 1A and FIG. 1B are flow charts of exemplary processes for preparing a liquid oat base or drink from an oat derived material.

FIG. 2 is a schematic view of a system for preparing a liquid oat base or drink from an oat derived material.

FIG. 3 is a schematic view of another example of a system for preparing a liquid oat base or drink from an oat derived material.

FIG. 4 is a schematic view of a heating device used for a system for preparing a liquid oat base or drink from an oat derived material.

### DETAILED DESCRIPTION

The present inventive concept will now be described more fully hereinafter with reference to the accompanying drawings, in which currently preferred variants of the inventive concept are shown. This inventive concept may, however, be implemented in many different forms and should not be construed as limited to the variants set forth herein; rather, these variants are provided for thoroughness and completeness, and fully convey the scope of the present inventive concept to the skilled person.

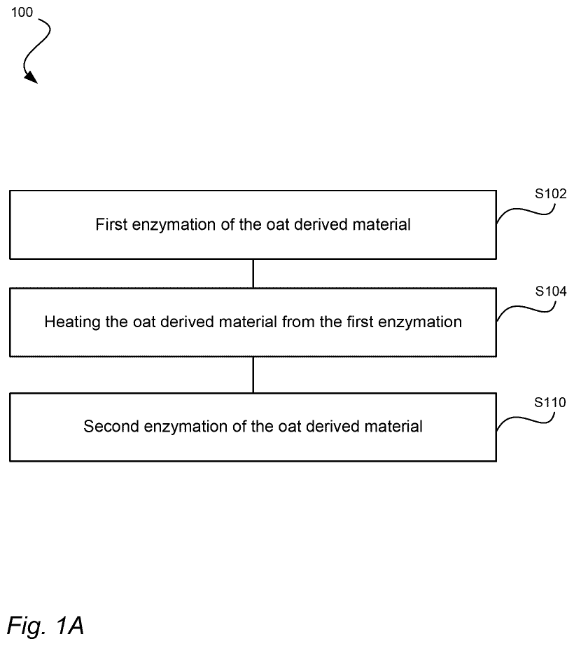

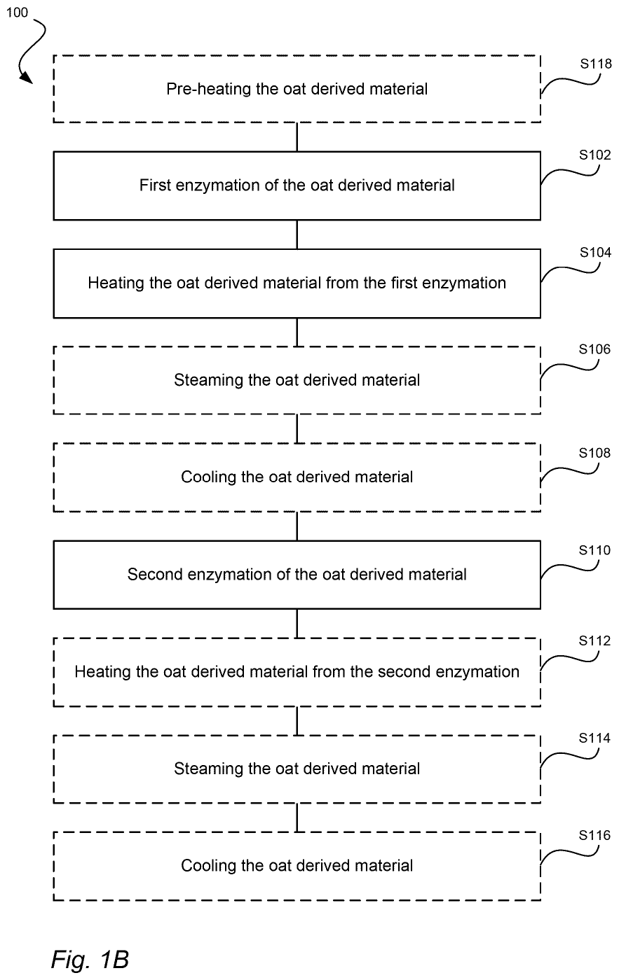

FIG. 1A is a flow chart illustrating steps of a process 100 for preparing a liquid oat base or drink from an oat derived material. Below, the different steps are described in more detail and starting with a first enzymation S102 of the oat derived material. The oat derived material may comprise micronized oats or micronized oat-kernels, or oat-flour. The oat derived material may also be provided as an aqueous or water suspension or slurry. The first enzymation S102 may comprise contacting the oat derived material with a starch degrading enzyme. The starch degrading enzyme may preferably be amylase. Alternatively, or in addition, the first enzymation S102 may comprise contacting the oat derived material with a protein solubilizing enzyme. The protein solubilizing enzyme may preferably be a protein deamidase such as protein glutaminase.

After the first enzymation S102 has been performed for a desired time, such as for example 30-90 minutes, preferably, 50-70 minutes, the oat derived material from the first enzymation S102 is heated S104 for a desired time, such as 1-15 minutes, preferably 3-7 minutes to terminate the enzymation thereof. The heating process may be such that the enzymation is either completely terminated or partly terminated. The heating process may have the effect of making the oat derived material more susceptible to enzymes. In other words, the oat derived material may be more accessible for enzymation.

For example, the heating of the oat derived material may gelatinize starch present in the oat derived material and allow swelling of starch and swelling of any present oat derived material granules, thereby providing open oat derived material. Open oat derived material may also be referred to as open oat particles, swelled starch, or hydrated starch.

It shall be understood that as oat derived material is treated in the process, for example by enzymes acting on the oat derived material, or the oat derived material being heated, the oat derived material typically changes, for example resulting from depletion of starch from the oat derived material. Oat derived material having undergone, for example the first enzymation S102, may therefore, herein, be referred to as oat derived material from the first enzymation S102, to differentiate it from e.g., oat derived material provided before the first enzymation S102. Oat derived material which has been subject to any degree of enzymation may alternatively be referred to as enzymised oat derived material.

Following the step of heating S104, a second enzymation S110 of the oat derived material from the heating S104 is performed. The second enzymation S110 may comprise contacting the oat derived material with a starch degrading enzyme, such as the same as in the first enzymation step S102, amylase. Alternatively, or in addition, the second enzymation S110 may comprise contacting the oat derived material with a protein solubilizing enzyme such as the same as in the first enzymation step S102, protein deamidase such as protein glutaminase.

From the discussion above, it is clear that a number of different combinations of enzymes are possible in the first and second enzymations S102, S110 for a duration of time such as for example 30-90 minutes, preferably, 50-70 minutes and which will be further discussed below.

In one example, the first and the second enzymation S102, S110 comprises contacting the oat derived material with the starch degrading enzyme and the protein solubilizing enzyme. Thus, enzymation involving solubilizing proteins may occur in two steps, both in the first enzymation S102 and in the second enzymation S110. This may allow for different types of protein solubilizing enzymes to be used at their respective optimum or suitable conditions, such as, for example, concerning temperature or substrate concentration which then can be customized at the different enzymation steps S102 and S110.

Further, the use of the protein solubilizing enzymes in the first and second enzymation steps S102 and S110 allows for increasing the overall efficiency of the protein solubilization enzymation. For example, protein solubilization in the first enzymation S102 may be performed to some degree already during the first duration of time for the first enzymation S102. Normally, less proteins are readily available for solubilization in the first enzymation S102 as compared to the second enzymation S110 when starch degradation has proceeded further but combined a total time of contact of the oat derived material with the protein solubilization enzyme is increased, thereby leaving a smaller number of proteins to be solubilized in the second enzymation S110 as compared to if no protein solubilization had taken place in the first enzymation S102.

Further, using the starch degrading enzyme and the protein solubilization enzyme in both the first and the second step S102 and S110 enables the same mixture of enzymes to be used for both steps, which allows for, for example, more efficient handling of the enzymes, a single source for providing and/or holding the enzymes.

In another example, the first and the second enzymation S102, S110 comprises contacting the oat derived material with the protein solubilizing enzyme and the first enzymation S102 further comprises contacting the oat derived material with the starch degrading enzyme. Similar benefits relating to using the protein solubilizing enzymes in both the first and second enzymation S102, S110 as mentioned above can be achieved, however absence of starch degradation in the second enzymation S110 may require that a desired level of starch degradation takes place in the first enzymation S102.

In another example, the first and the second enzymation S102, S110 comprises contacting the oat derived material with the protein solubilizing enzyme and the second enzymation S110 further comprises contacting the oat derived material with the starch degrading enzyme.

In yet another example, the first enzymation S102 comprises contacting the oat derived material with the starch degrading enzyme and the second enzymation S110 comprises contacting the oat derived material with the protein solubilizing enzyme. A desirable effect may be that the solubilizing of protein in the oat derived material can be focused to the second enzymation S110 where the oat derived material is most susceptible to the protein solubilizing enzyme due to the degradation of the oat derived material from the starch degrading enzyme in the first enzymation S102. The use of different enzymes for respective enzymation step S102 or S110 allows for customized and optimized temperatures for the protein solubilization enzyme and the starch degrading enzyme to be used.

In yet another example, the first enzymation S102 comprises contacting the oat derived material with the starch degrading enzyme and the second enzymation S110 comprises contacting the oat derived material with both the starch degrading enzyme and the protein solubilizing enzyme. The same desirable and synergistic effect as the previous example may be achieved, as well as achieving further swelling of the oat derived material by the addition of the starch degrading enzymes in the second enzymation S110 as well.

In yet another example, the first enzymation S102 comprises contacting the oat derived material with the starch degrading enzyme and the protein solubilizing enzyme and the second enzymation S110 comprises contacting the oat derived material with the starch degrading enzyme.

It should be noted that even though the same enzymes may be used in the first and second enzymation S102, S110, the amount of enzyme in relation to the amount of oat derived material may be the same or different.

Further, the ratio between the amount of either enzyme may be the same or different. For example, more enzyme may be added in the first enzymation S102 than the second enzymation S110, or the opposite. Similarly, a time period in which the oat derived material is enzymised in the first enzymation S102 and the second enzymation S110 respectively may be different or the same.

Illustrated in FIG. 1B is another example of the processes 100 for preparing the liquid oat base or drink from the oat derived material wherein the oat derived material from the heating S104, may be cooled S108. The cooling S108 of the oat derived material from the heating S104 may be performed after the heating S104 of the oat derived material and before the second enzymation S110 of the oat derived material. In other words, the heated enzymized oat derived material from the first enzymation S102 may be cooled S108.

The oat derived material, from the heating S104, may be steamed S106. The steaming S106 of the oat derived material may be performed after the heating S104 of the oat derived material and before the cooling S108 of the oat derived material. In other words, the enzymised oat derived material may be injected with steam to increase the heat of the oat derived material from the first enzymation S102. Thus, further terminating the enzymation.

The oat derived material, from the second enzymation S110, may be heated a second time S112. The second heating S112 may be performed after the second enzymation S110 of the oat derived material. Put differently, the enzymised oat derived material from the second enzymation S112 may also be heated to terminate the enzymation thereof.

The oat derived material, from the second heating S112, may be cooled a second time S116. The second cooling S116 of the oat derived material may be performed after the second heating S112 of the oat derived material. In other words, the heated enzymized oat derived material S112 from the second enzymation S110 may be cooled S116.

The oat derived material, from the second heating S112, may be steamed a second time S114. The second steaming S114 of the oat derived material may be performed after the second heating S112 of the oat derived material and before the second cooling S116 of the oat derived material. In other words, the enzymised oat derived material may be injected with steam to increase the heat of the oat derived material from the second enzymation S110. Thus, further terminating the enzymation of the second enzymation S110.

Thus, the heating S104, S110 and/or steaming S106, S114 and/or cooling S108, S116 of the oat derived material may be performed at the first enzymation S102, the second enzymation S110 or at both the first enzymation S102 and the second enzymation S110. The heating S104, S110 and/or steaming S106, S114 may be performed at a same temperature or at different temperatures. In some examples the temperature is chosen based on a desired temperature to deactivate enzymation partly or fully.

The oat derived material may be heated in a pre-heating step S118, before the first enzymation S102. The heat treatment of the oat derived material in the pre-heating step S118 may be milder than the first and/or second heating S104, S112. In other words, the oat derived material may be heated to a lower temperature during the pre-heating S118, than during the first and/or second heating S104, S112.

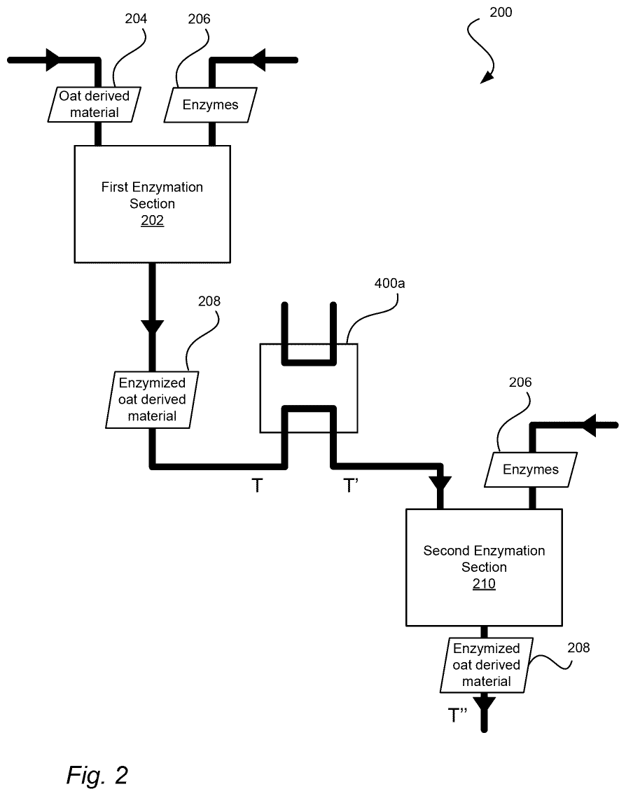

FIG. 2 schematically illustrates one example of a system 200 for preparing a liquid oat base or drink from an oat derived material.

The system 200 may comprise a first enzymation section 202. The first enzymation section 202 may be adapted for a first enzymation of the oat derived material. The first enzymation section 202 may thus be configured to receive the oat derived material 204 and enzyme 206. Put differently, the first enzymation section 202 may be fluidly connected to a supply (not shown) of oat derived material. Further, the first enzymation section 202 may be fluidly connected to a supply (not shown) of enzyme. The enzyme 206 may be added to the oat derived material 204 prior to entering the first enzymation section 202. Thus, the first enzymation section 202 may have one inlet.

The first enzymation section 202 may comprise a conduit or a reservoir such as a tank. The first enzymation section 202 may comprise two or more conduits or reservoirs such as tanks.

The first enzymation section may output enzymised oat derived material 208.

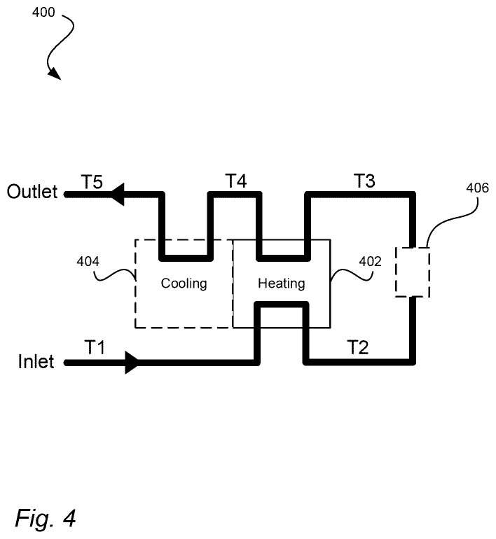

The system 200 further comprise a heating device 400a. The heating device 400a may be fluidly connected to the first enzymation section 202. The heating device 400a is arranged downstream from the first enzymation section 202. Thus, the heating device 400a receives the enzymised oat derived material 208 outputted from the first enzymation section 202. The heating device 400a heats the oat derived material 208 from the first enzymation section 202. The heating device 400a may heat the oat derived material from a first temperature T to a second temperature T′. The heating device 400a may heat the oat derived material to terminate the enzymation process. The heating device 400a is further described in connection to FIG. 4.

The system 200 may further comprise a second enzymation section 210. The second enzymation section 210 is fluidly connected to the heating device 400a. The second enzymation section 210 is arranged downstream of the heating device 400a. The second enzymation section 210 is adapted for a second enzymation of the enzymised oat derived material 208. The second enzymation section 210 may thus be configured to receive oat derived material from the heating device 400a and enzyme from a supply of enzyme (not shown). Even though the inlet of the enzyme 206 is illustrated as separate from the enzymised oat derived material, they may be combined. Put differently, the enzyme 206 may be mixed with the oat derived material prior to entering the second enzymation section 210. The supply of enzyme for the second enzymation section 210 may also be the same as the supply for the first enzymation section 202.

The second enzymation section 210 outputs a second enzymised oat derived material. The enzymised oat derived material outputted from the second enzymation section 210 may be enzymised to a higher degree than the enzymised oat derived material outputted from the first enzymation section 202. Alternatively, the enzymised oat derived material outputted from the second enzymation section 210 may be enzymised in a different way from the enzymised oat derived material outputted from the first enzymation section 202.

The second enzymation section 210 may comprise a conduit or a reservoir such as a tank. The second enzymation section 210 may comprise two or more conduits or reservoirs such as tanks.

It shall be realized and appreciated that the illustration of FIG. 2 is a simplified view for illustrative purposes. Other components may be part of the system 200 as well, which would be apparent to the skilled person, even though not illustrated. For example, valves, sensors, pipe connections, controllers, etc.

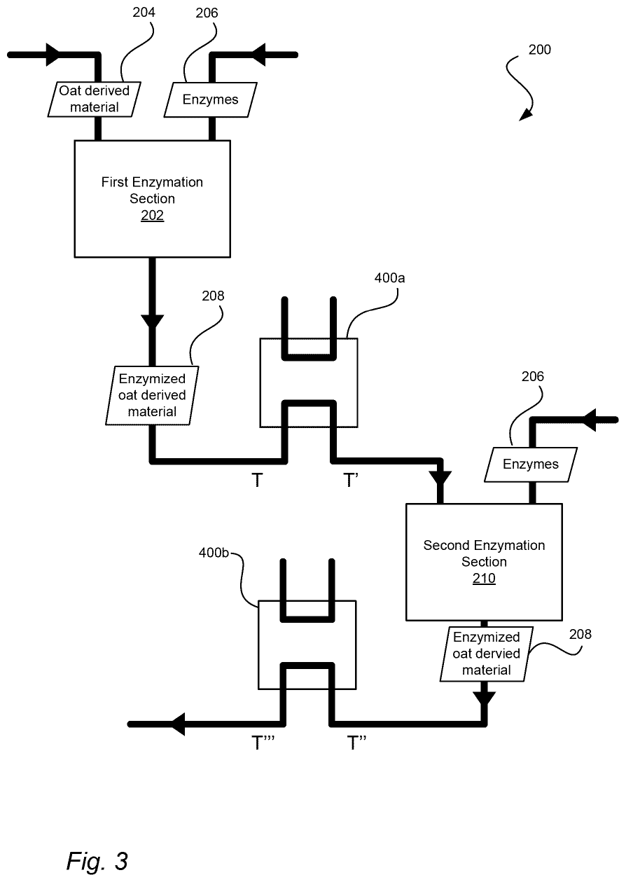

FIG. 3 schematically illustrates another example of the system 200 for preparing the liquid oat base or drink from the oat derived material.

In addition to what is described in connection to FIG. 2, the example of the system 200 illustrated in FIG. 3 may further comprise a second heating device 400b. The second heating device 400b is fluidly connected to the second enzymation section 210. The second heating device 400b may be arranged downstream from the second enzymation section 210. The second heating device 400b is adapted to heat the oat derived material a second time. The oat derived material may be heated from a third temperature T″ to a fourth temperature T″′.

FIG. 4 schematically illustrates the heating device 400 in the system 200 for preparing the liquid oat base or drink from the oat derived material. The heating device 400 illustrated herein may thus constitute the first heating device 400a and/or second heating device 400b illustrated in FIG. 2 and/or FIG. 3. The heating device 400 may change the temperature of the oat derived material from a temperature T1 to a temperature T5. The temperature T5 may be higher than the temperature T1. Alternatively, the temperature T5 may be the same as the temperature T1. Alternatively, the temperature T5 may be lower than the temperature T1.

The heating device 400 may comprise a heat exchanger 402. The heat exchanger may increase the temperature of the oat derived material from the temperature T1 to a temperature T2. The heating device 400 may further comprise a cooling section 404. The cooling section 404 may be a part of the heat exchanger 402, or separate. Thus, the heating device 400 may be adapted to both heat the oat derived material and cool the oat derived material. The cooling section 404 may reduce the temperature of the oat derived material from a temperature T4 to the temperature T5. Thus, even though the temperature T1 of the inputted oat derived material may be the same as, or lower than, the temperature T5 of the outputted oat derived material, the oat derived material may still have been heated to a higher temperature as some point thus terminating the enzymation of the oat derived material.

The heating device 400 may comprise a steam injector 406. The steam injector 406 is fluidly connected to the heat exchanger 402 and arranged downstream from the temperature change T1 to T2 of the heat exchanger 402 but before the change of the temperature T3 to T4 of the heat exchanger 402. The steam injector 406 may be adapted to steam the oat derived material, thus increasing the temperature of the oat derived material from the temperature T2 to the temperature T3.

As illustrated herein, the heat exchanger 402 may be arranged to exchange heat between the oat derived material before the steam injector 406 and the oat derived material after the steam injector 406. This may be advantageous in that heat energy produced by the steam injector can be re-used in the heating and/or cooling of the oat derived material from the temperature T1 to the temperature T2 and/or T4 to T5, thus saving energy. Further, the oat derived material from the steam injector 406 can be cooled from the temperature T3 to the temperature T4, thus reducing the amount of cooling needed by the cooling section 404.

The heating device 400 is not limited to comprising a heat exchanger and/or steam injector 406. The heating device 400 may be any device suitable for the cause. For example, the heating device 400 may comprise a device for heating the oat derived material by using microwaves. The heating device 400 may be an immersion heater. The heating device may comprise tubular heating elements. The heating device 400 may be a circulation heater. The heating device 400 may be an electrode heater.

The cooling section 404 may be a heat exchanger connected to a cooling medium.

The heating process may take approximately 3-10 minutes, such as 4-7 minutes. The time of the heating process in the first and/or second heating device 400a, 400b may be the same or different.

The temperatures T1-T5 may be different in the first heating device 400a and the second heating device 400b.

For example, in the first heating device 400a, the temperature T5 may be 60 degrees C. The temperature T5 may correspond to the second temperature T′ or T″ as illustrated in FIG. 2. The temperature T3 may then for example be 110 degrees C.

For example, in the second heating device 400b, the temperature T5 may be 5 degrees C. The temperature T5 may in this case correspond to the fourth temperature T″′ as illustrated in FIG. 3. The temperature T3 may then for example be 90 degrees C.

The Temperatures T1-T5 may be selected based on for example a desired enzyme activation and/or deactivation such as the starch degrading enzyme and/or the protein solubilizing enzyme, and the temperatures T1-T5 may be selected as, Temperature T1 may be 55 to 65 degrees C., or more preferably 60 degrees C. Temperature T2 may be 75 to 85 degrees C., or more preferably 80 degrees C. Temperature T3 may be 85 to 95 degrees C., or more preferably 90 degrees C. Temperature T4 may be 65 to 75 degrees C., or more preferably 70 degrees C. Temperature T5 may be 55 to 65 degrees, or more preferably 60 degrees C.

Reference is hereby made to three related applications with the same applicant and filing date as the present application and disclosing variants and related methods and systems as herein disclosed for preparing a liquid oat base or drink from an oat derived material. A related application titled A BATCH PROCESS AND SYSTEM FOR PREPARING A LIQUID OAT BASE OR DRINK FROM AN OAT DERIVED MATERIAL discloses a process and system for preparing a liquid oat base or drink from an oat derived material where the first and/or second enzymation sections comprises two or more enzymation tanks respectively for higher throughput. Another related application titled A CONTINOUS PROCESS AND SYSTEM FOR PREPARING A LIQUID OAT BASE OR DRINK FROM AN OAT DERIVED MATERIAL discloses a process and system for preparing a liquid oat base or drink from an oat derived material where the first and/or second enzymation sections comprises a first and second volume respectively with continuous inlet and outlet for higher throughput. Another related application titled A MIXED PROCESS AND SYSTEM FOR PREPARING A LIQUID OAT BASE OR DRINK FROM AN OAT DERIVED MATERIAL discloses a process and system for preparing a liquid oat base or drink from an oat derived material where one of the first and second enzymation sections comprises two or more enzymation tanks and the other of the first and second enzymation section comprises a volume with continuous inlet and outlet for higher throughput.

It is understood that other variations in the present invention are contemplated and, in some instances, some features of the invention can be employed without a corresponding use of other features. Accordingly, it is appropriate that the appended claims be construed broadly in a manner consistent with the scope of the invention.

1. A process (100) for preparing a liquid oat base or drink from an oat derived material, comprising:
a first enzymation (S102) of the oat derived material,
heating (S104) the oat derived material from the first enzymation (S102) to terminate the enzymation thereof, and
a second enzymation (S110) of the oat derived material from the heating (S104),
wherein the first and/or the second enzymation (S102, S110) comprises contacting the oat derived material with starch degrading enzyme, and
wherein the first and/or second enzymation (S102, S110) further comprises contacting the oat derived material with protein solubilizing enzyme.

2. The process (100) according to claim 1, further comprising cooling (S108) the oat derived material after the step of heating (S104) the oat derived material and before the step of the second enzymation (S110) of the oat derived material.

3. The process (100) according to claim 1, further comprising a step of steaming (S106) the oat derived material after the step of heating (S104) the oat derived material to terminate the enzymation and before the step of cooling (S108) the oat derived material.

4. The process (100) according to claim 1, further comprising a step of heating (S112) the oat derived material a second time to terminate the enzymation thereof, after the step of the second enzymation (S110) of the oat derived material.

5. The process (100) according to claim 1, further comprising a step of cooling (S116) the oat derived material a second time after the step of heating (S112) the oat derived material the second time.

6. The process (100) according to claim 1, further comprising a step of steaming (S114) the oat derived material a second time, after the step of heating (S112) the oat derived material the second time and before the step of cooling (S116) the oat derived material the second time.

7. The process (100) according to claim 1, wherein the oat derived material comprises micronized oats or micronized oat-kernels, oat-flour, or fractions of oat.

8. The process (100) according to claim 1, wherein the oat derived material is provided as an aqueous or water suspension or slurry.

9. The process (100) according to claim 1, wherein the oat derived material starch degrading enzyme is amylase.

10. The process according to claim 1, wherein
the oat derived material protein solubilizing enzyme is protein deamidase or protein glutaminase. oat derived material.

11. A system (200) for preparing a liquid oat base or drink from an oat derived material (204), comprising:
a first enzymation section (202) adapted for a first enzymation of the oat derived material (204),
a heating device (400a) fluidly connected to and arranged downstream of the first enzymation section (202) and adapted to heat the oat derived material (208) from the first enzymation, and
a second enzymation section (210) fluidly connected to and arranged downstream of the heating device (400a) and adapted for a second enzymation of the oat derived material (208) from the heating device (400a).

12. The system (200) according to claim 11, wherein the heating device (400a) is adapted to both heat the oat derived material (208) in a first step and thereafter cool the oat derived material (208) in a second step.

13. The system (200) according to claim 11, wherein the heating device (400a) comprises a heat exchanger and/or a steam injector (406).

14. The system (200) according to claim 11, further comprising a second heating device (400b) fluidly connected to and arranged downstream of the second enzymation (210) section and adapted to heat the oat derived material (208) a second time.

15. The system (200) according to claim 14, wherein the second heating device (400b) is adapted to both heat the oat derived material (208) in a first step and thereafter cool the oat derived material (208) in a second step.

16. The system (200) according to claim 14, wherein the second heating device (400b) comprises a heat exchanger and/or a steam injector (406).

17. The system (200) according to claim 11, wherein the first enzymation section (202) and the second enzymation section (210) comprises a conduit or a reservoir, such as a tank, respectively.
